(19) United States
(12) Patent Application Publication
(10) Pub. No.: US 2007/0004974 A1
(43) Pub. Date: Jan. 4, 2007

(54) Glucometer comprising an implantable light source

(75) Inventors: Ron Nagar, Tel-Aviv (IL); Gabriel Bitton, Jerusalem (IL); Benny Pesach, Rosh-Ha'Ayin (IL)

Correspondence Address: WOLF, BLOCK, SCHORR & SOLIS-COHEN LLP, 250 PARK AVENUE, NEW YORK, NY, 10177, US

(73) Assignee: Glucon, Inc., Boulder, CO (US)

(21) Appl. No.: 10/596,939

(22) PCT Filed: Dec. 23, 2004
(86) PCT No.: PCT/IL04/01166

§ 371 Date: Jun. 29, 2006

Related U.S. Application Data
(60) Provisional application No. 60/532,573, filed on Dec. 29, 2003.

(51) Int. Cl. A61B 5/00; A61B 8/00

(52) U.S. Cl. 600/316; 600/310; 600/438

(57) ABSTRACT

Apparatus for assaying an analyte in a body comprising: at least one light source implanted in the body controllable to illuminate a tissue region in the body with light at at least one wavelength that is absorbed by the analyte and as a result generates photoacoustic waves in the tissue region; at least one acoustic sensing transducer coupled to the body that receives acoustic energy from the photoacoustic waves and generates signals responsive thereto; and a processor that receives the signals and processes them to determine a concentration of the analyte in the illuminated tissue region.

### RELATED APPLICATIONS

The present application claims the benefit under 35 USC 119(e) of U.S. provisional application 60/532,573 filed on Dec. 29, 2003, the disclosure of which is incorporated herein by reference.

### FIELD OF THE INVENTION

The invention relates to apparatus for assaying a substance in a body and in particular to apparatus that comprises a light source implanted in the body for assaying a body analyte.

### BACKGROUND OF THE INVENTION

Methods and apparatus for determining blood glucose levels for use in the home, for example by a diabetic who must monitor blood glucose levels frequently, are available. These methods and associated devices are generally invasive and usually involve taking blood samples by finger pricking. Often, a diabetic must determine blood glucose levels many times daily and finger pricking is perceived as inconvenient and unpleasant. To avoid finger pricking diabetics tend to monitor their glucose levels less frequently than is advisable.

In addition, prior art glucose assaying methods and devices, such as those based on finger pricking, are generally not suitable or cannot provide substantially continuous monitoring of a patient's glucose level. Continuous monitoring is advantageous for reducing delay from a time at which a change in blood glucose level occurs that demands patient intervention to a time at which the patient is alerted to the change. Continuous monitoring would also be particularly advantageous for use with drug delivery devices for automatic delivery of drugs to a patient to control the patient's glucose levels.

US Patent Application Publication 2003/0023317, the disclosure of which is incorporated herein by reference, describes a device, hereinafter a “glucometer”, for assaying glucose that does not require finger pricking and may provide continuous monitoring of a patient's glucose levels. The glucometer is implantable and comprises a sensing membrane that includes an enzyme for detecting and assaying the patient's glucose. The glucometer comprises a bio-interface membrane overlaying the sensing membrane that promotes vascularization of tissue in a layer of the bio-interface membrane and enables glucose in the patient's body to reach and contact the enzyme in the sensing membrane. However, enzymes used in glucometers generally require periodic renewal to maintain their efficacy and therefore limit a period of time for which the implanted glucometer may be unattended.

Optical methods and devices based on optical methods for assaying glucose appear to be advantageous for long term convenient in vivo monitoring of a patient's glucose levels. Glucometers that use optical assay methods, such as for example near infrared (NIR) or mid infrared (MIR) absorption and/or scattering spectroscopy methods, do not generally require chemical interaction of glucose with another substance in order to determine concentration of a patient's glucose. Optical methods are therefore usually minimally or non-interactive with the patient's metabolism and generally do not require that a reagent, which might have to be periodically renewed, be introduced into the patient's body to assay his or her glucose.

However, light at any given wavelength in the NIR and MIR wavelength bands, is generally not “specific” to glucose (or any other particular analyte in the body) interacting substantially only with glucose and at most a very few additional analytes. Light at an NIR or MIR wavelength generally interacts with water and many other substances in the body besides glucose, such as urea, albumin, hemoglobin and uric acid, which in general absorb and/or scatter the light. Water in particular is a very strong absorber of light in the NIR and MIR wavelength bands and in general dominates absorption of light in these wavelength bands. As a result, determining a patient's glucose concentration from optical absorption and/or scattering processes generally requires acquiring measurements at different wavelengths of light and relatively complicated multivariate analysis of the measurements.

Wavelengths, hereinafter “signatory wavelengths”, do exist in the NIR and MIR bands that are highly specific to glucose and have for a given tissue type substantial absorption cross-sections only for glucose and water in the tissue. Such wavelengths are potentially useable to assay glucose without requiring complicated procedures for acquiring and analyzing a relatively large number of absorption and/or scattering measurements at a plurality of different wavelengths. However, absorption of light by water at signatory wavelengths is generally so strong, that light at these wavelengths attenuates rapidly in living tissue. When a region of a person's skin is illuminated by light at the wavelengths, the light does not generally have a useful penetration depth greater than about 30 to about 50 microns for acquiring absorption measurements. For these penetration depths, glucose measurements are generally inaccurate as indicators of a patient's glucose concentration.

For example, an article by Martin, W. B., et al entitled “Using two discrete frequencies within the middle infrared to quantitatively determine glucose in serum”; Journal of Biomedical Optics; October 2002; Vol. 7, No. 4, pp 613-617, notes that light at wavelengths 9.66 microns (wave number 1035 cm⁻¹) and 9.02 microns (wave number 1109 cm⁻¹) can be advantageous for assaying glucose. Light at “signatory wavelength” 9.66 microns is strongly absorbed substantially only by glucose and water in living tissue and may be advantageous for assaying glucose in blood. Light at wavelength 9.02 microns is strongly absorbed substantially only by glucose, hemoglobin and water in living tissue and may be advantageous for assaying glucose in interstitial fluid, which does not in general comprise hemoglobin, and for which therefore 9.02 microns is a signatory wavelength and in blood The effect of absorption of light by water at these wavelengths may generally be removed relatively straightforwardly from absorption measurements of light at the wavelengths. The article suggests that light at a wavelength of 9.66 microns may be advantageous for use in an implantable glucometer comprising both a light source and a photodetector for the light. The article does not provide details or describe a configuration of the proposed implantable glucometer.

U.S. Pat. No. 6,049,727, the disclosure of which is incorporated herein by reference, describes an in vivo sensor for determining concentration of a constituent of a patient's body fluid comprising an optical source and photodetector that are implanted in the patient's body with the optical source and photodetector straddling a vein. The patent describes the vein as being between 0.3 and 1.0 mm in diameter. The source and photodetector are controllable to acquire absorption measurements at a plurality of wavelengths of light at least one of which is in the IR band. For determining glucose concentration, the diameter of the vein and distances between the vein wall and the light source and photodetector appear to preclude use of light at a signatory wavelength of glucose. The patent suggests that for assaying glucose, absorption measurements at a relatively large number of about thirteen different wavelengths of light should be acquired. Whereas the light source and photodetector are implanted in the body, the patent notes that components of the sensor, such as a processor, may be located external to the body.

PCT Application number PCT/IL2004/000483 filed on Jun. 8, 2004 by some of the same inventors as the present invention, the disclosure of which is incorporated herein by reference, describes a wearable glucometer that provides real time in-vivo assays of glucose in blood in a patient's blood vessel. The glucometer transmits light through the patient's skin at an intensity and wavelength for which light penetrates body tissue and illuminates blood in the blood vessel with an amount of light that stimulates photoacoustic waves in the blood vessel having sufficient intensity so that they are useable by the glucometer to assay glucose.

### SUMMARY OF THE INVENTION

An aspect of some embodiments of the present invention relates to providing a glucometer that can provide relatively long term monitoring of a patient's glucose level without requiring substantial user attention or intervention.

An aspect of some embodiments of the present invention relates to providing a glucometer comprising a light source implanted in a patient's body that assays the patient's glucose responsive to interaction of light provided by the light source with body tissue.

According to an aspect of some embodiments of the invention, the light interacts with the body tissue to generate photoacoustic waves in the tissue and the glucometer determines glucose concentration responsive to the photoacoustic waves.

In an embodiment of the invention, the implanted light source is sealed in a suitable capsule having an optical aperture substantially transparent to light provided by the light source. Optionally, the light source provides light at a signatory wavelength of glucose. Optionally, the aperture is covered with a membrane that tends to prevent formation of a barrier cell layer of inflammatory response cells (e.g. macrophages) over the aperture and promotes vascularization and presence of interstitial fluid comprising glucose close to the aperture. Optionally, the at least one bio-membrane promotes vascularization at distances from the aperture that are less than a small number (for example, less than 1, 2 or 3) of extinction lengths of light provided by the light source in the patient's body tissue. Light provided by the light source is therefore not substantially attenuated before it interacts with the patient's interstitial fluid and/or blood and generates photoacoustic waves therein that are a function of the patient's glucose concentration. The patient's glucose is assayed responsive to the photoacoustic waves.

In some embodiments of the invention, the optical aperture is not covered with a bio-membrane that prevents formation of a barrier cell layer of inflammatory response cells over the aperture. As a result, after insertion of the capsule in the body of the patient a barrier cell layer forms over the aperture. In accordance with an embodiment of the invention, photoacoustic waves generated in the barrier cell layer are used to assay glucose in the barrier cell layer and provide an assay of the patient's glucose.

In some embodiments of the invention, the light source is enclosed in a capsule formed so that it comprises a sample volume bordered by at least one membrane that is permeable to interstitial fluid and glucose therein. As a result, the sample volume fills with interstitial fluid from the patient's body. Light from the light source illuminates the interstitial fluid in the sample volume and generates photoacoustic waves therein that are used to assay glucose in the fluid and provide an assay of the patient's glucose.

In some embodiments of the invention, an acoustic transducer external to the body is coupled to the patient's skin and generates signals responsive to the photoacoustic waves that are processed to determine the patient's glucose concentration.

In some embodiments of the invention, the capsule comprises an antenna for receiving electromagnetic energy and an external transmitter transmits electromagnetic waves to the antenna to power the light source.

In some embodiments of the invention the capsule comprises an acoustic transducer and an external transmitter transmits acoustic waves to the transducer to power the light source.

An aspect of some embodiments of the present invention relates to providing a glucometer comprising an implanted sensor unit that provides assays of a patient's glucose responsive to attenuation of light in the patient's interstitial fluid According to an embodiment of the invention the sensor unit comprises a light source and a photosensor that sandwiches between them a “test” volume, which is bordered by at least one membrane that is permeable to interstitial fluid and glucose therein. The light source transmits light into the test volume towards the photosensor. Glucose in interstitial fluid in the test volume absorbs energy from the light and attenuates the light. Signals generated by the photosensor responsive to intensity of light that propagates through the test volume are a measure of the attenuation that the light undergoes in the test volume. The glucometer determines an assay of the patient's glucose responsive to the signals.

There is therefore provided in accordance with an embodiment of the invention, apparatus for assaying an analyte in a body comprising: at least one light source implanted in the body controllable to illuminate a tissue region in the body with light at at least one wavelength that is absorbed by the analyte and generates thereby photoacoustic waves in the tissue region; at least one acoustic sensing transducer coupled to the body that receives acoustic energy from the photoacoustic waves and generates signals responsive thereto; and a processor that receives the signals and processes them to determine a concentration of the analyte in the illuminated tissue region.

Optionally, the apparatus comprises: at least one acoustic transmitting transducer coupled to the body controllable to transmit ultrasound, a controller adapted to control the at least one transmitting transducer; wherein the controller controls the transmitting transducer to transmit ultrasound that is incident on the illuminated tissue region and thereafter on the at least one sensing transducer and the processor processes signal generated by the sensing transducer responsive to the ultrasound to determine a change in an acoustic property of the tissue region caused by the illumination and therefrom an assay of the analyte.

There is further provided in accordance with an embodiment of the invention, apparatus for assaying an analyte in a body comprising: at least one light source implanted in the body controllable to illuminate a tissue region in the body with light at at least one wavelength that is absorbed by the analyte and generates a change in an acoustic property of the region; at least one sensing acoustic transducer that generates signals responsive to acoustic energy incident thereon; at least one transmitting acoustic transducer that transmits ultrasound that is incident on the region and thereafter on the sensing transducer; and a processor that receives signals generated by the sensing transducer responsive to the incident ultrasound and processes them to determine a measure of the change and therefrom concentration of the analyte.

There is further provided in accordance with an embodiment of the invention, apparatus for assaying an analyte in a body comprising: a membrane formed from a material, permeable to interstitial fluid in the body and the analyte therein that bounds a volume region in the body, which volume region contains interstitial fluid that permeates through the membrane to enter the volume; at least one light source implanted in the body that illuminates the volume with light at at least one wavelength that is absorbed by the analyte and generates thereby photoacoustic waves in the volume; at least one acoustic sensing transducer coupled to the body that receives acoustic energy from the photoacoustic waves and generates signals responsive thereto; and a processor that receives the signals and processes them to determine a concentration of the analyte in the illuminated volume.

There is further provided in accordance with an embodiment of the invention, apparatus for assaying an analyte in interstitial fluid in a body comprising: at least one light source implanted in the body that provides light at at least one wavelength that is absorbed by the analyte; at least one photosensor implanted in the body that receives light from the at least one light source and generates signals responsive thereto; a membrane formed from a material permeable to components of interstitial fluid in the body and the analyte that bounds a volume sandwiched between the at least one light source and the at least one photosensor and wherein the light from the at least one light source that reaches the at least one photosensor propagates through the volume; and circuitry that receives the signals from the at least one photosensor and uses them to provide an assay of the analyte in the body.

Optionally, a gap between the source and the sensor is 10 to 50 micrometers. Optionally, a gap between the source and the sensor is 50 to 150 micrometers.

In some embodiments of the invention, the absorption coefficient for light in tissue in the region at a wavelength of the at least one wavelength is substantially equal to the sum of the absorption coefficients of the analyte and at most a relatively small number of additional analytes. Optionally, the at least one additional analyte comprises a number of analytes less than or equal to three. Optionally, the at least one additional analyte comprises a number of additional analytes less than or equal to two. Optionally, the at least one additional analyte comprises one additional analyte. In some embodiments of the invention, the at least one additional analyte comprises water.

In some embodiments of the invention, the at least one implanted light source is encapsulated in a capsule having an aperture substantially transparent to light at the at least one wavelength through which light is transmitted to illuminate the region.

Optionally, the apparatus comprises a layer of a biocompatible material overlaying the aperture that promotes vascularization of tissue in close proximity to the aperture and wherein the region comprises the vascularized tissue. Optionally, vascularization is promoted at distances from the aperture that are less than an extinction length for light at the at least one wavelength.

Additionally or alternatively, the capsule comprises a receiver for receiving energy to power the at least one light source. Optionally, the receiver comprises an antenna for receiving electromagnetic energy. Optionally, the apparatus comprises a transmitter external to the body that transmits electromagnetic energy to the antenna.

In some embodiments of the invention, the receiver comprises an acoustic transducer for receiving acoustic energy. Optionally, the apparatus comprises an acoustic transmitter external to the body that transmits acoustic energy to the acoustic transducer comprised in the receiver.

In some embodiments of the invention, the analyte is glucose. In some embodiments of the invention, the at least one wavelength comprises a wavelength equal to about 9.66 microns. In some embodiments of the invention, the at least one wavelength comprises a wavelength equal to about 9.02 microns. In some embodiments of the invention, the at least one wavelength comprises a plurality of wavelengths.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 shows a glucometer comprising an implanted light source, in accordance with an embodiment of the present invention;

FIG. 2 schematically shows the implanted light source shown in FIG. 1 being implanted using a syringe, in accordance with an embodiment of the present invention;

FIG. 3 schematically shows another glucometer comprising an implanted light source, in accordance with an embodiment of the present invention; and

FIG. 4 schematically shows a glucometer comprising an implanted sensor unit that provides measures of attenuation of light in interstitial fluid in a patient's body and therefrom assays of the patient's glucose in accordance with an embodiment of the invention.

### DETAILED DESCRIPTION OF EXEMPLARY EMBODIMENTS

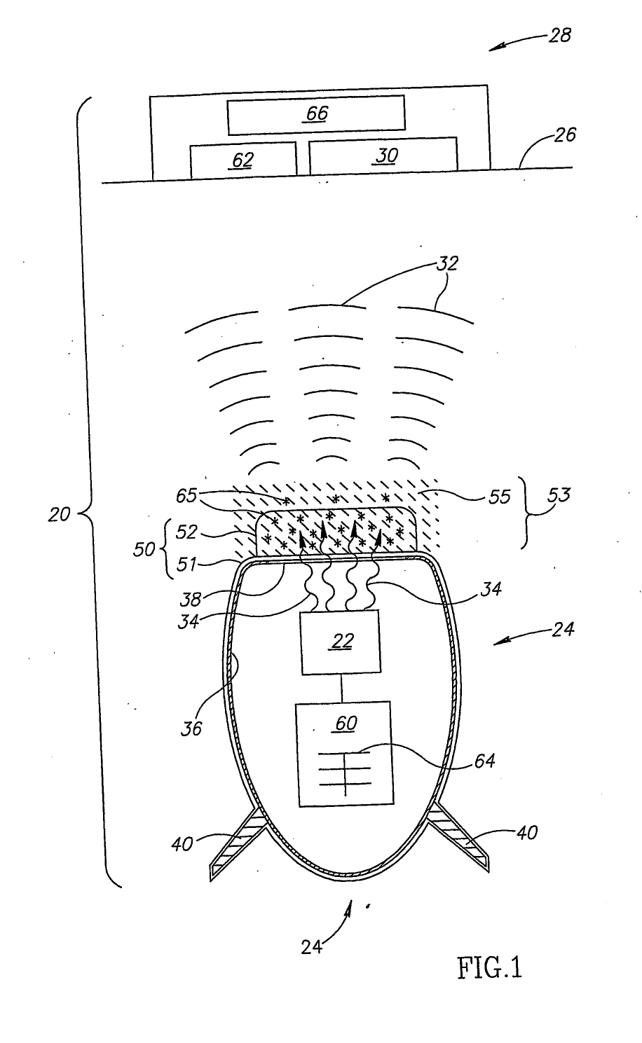

FIG. 1 schematically shows a glucometer 20, comprising a light source 22 encapsulated in a capsule 24, which is implanted below the skin 26 of a patient and a control unit 28 comprising at least one acoustic transducer 30, in accordance with an embodiment of the invention. Glucometer 20 is schematically shown determining the patient's glucose concentration responsive to photoacoustic waves, schematically represented by arcs 32, that are generated in the patient's body by light 34 from light source 22.

Capsule 24 comprises a casing 36 formed from a suitable material, such as a biocompatible metal or plastic, and has an optical aperture 38 through which light 34 may exit the casing. Capsule 24 is optionally oriented so that optical aperture 38 faces at least one transducer 30. Capsule 24 optionally comprises a plurality of anchor stubs 40 that aid in stabilizing the location and orientation of the capsule in the patient's body. A method of inserting capsule 24 below the patient's skin and deploying anchor stubs 40 is discussed below.

Aperture 38 is optionally covered by a membrane 50 comprising a bio-sealing layer 51 and a bio-promoting layer 52 overlaying the bio-sealing layer that is substantially transparent to light provided by light source 22, is resistant to cellular attachment and is substantially impermeable to cells and substances they produce. Optionally, bio-sealing layer 51 covers substantially all of casing 36. Bio-sealing layer 51 functions to substantially seal off material that it covers from the body's biological processes. Thickness of bio-sealing layer 51 is such that attenuation of light 34 in the bio-sealing layer does not prevent the light from stimulating photoacoustic waves 32 having sufficient intensity so that they are useable for assaying the patient's glucose in accordance with an embodiment of the invention. Thickness of bio-sealing layer 51 is optionally less than about 1 or 2 extinction lengths of light 34 in the layer.

Bio-promoting layer 52 is configured and formed from a material that promotes vascularization therein. Optionally, bio-promoting layer 52 overlays bio-sealing layer 51 substantially only where the bio-sealing layer overlays aperture 38. Membrane 50 comprising bio-sealing and bio-promoting layers 51 and 52 tends to prevent formation of a barrier cell layer of inflammatory response cells (e.g. macrophages, fibroblasts and giant cells) over aperture 38 that attenuates light 34. The membrane also promotes growth of vascularized tissue comprising interstitial fluid and blood in close proximity to aperture 38 in and adjacent to bio-promoting layer 52. A shaded region 53 indicates a vascularized tissue region comprising interstitial fluid located in bio-promoting layer 52 and in a tissue region 55 in proximity to the layer.

Suitable materials and structures for producing bio-sealing and bio-promoting layers 51 and 52 comprised in membrane 50 and a manner in which they operate to promote growth of vascularized tissue adjacent to a surface of an artificial insert are described in US Patent Application Publication 2003/0023317 cited above. Optionally, bio-sealing layer 51 is made of parylene and bio-promoting layer 52 is a layer, having voids, that is optionally formed from silicone.

Capsule 24 optionally comprises circuitry 60 for controlling light source 22 and providing energy to operate the light source. Optionally, circuitry 60 is powered by energy that it receives from a transmitter 62, optionally located in control unit 28, and comprises a receiver 64 for receiving the energy. Optionally, circuitry 60 comprises a device (not shown) for storing energy, such as a capacitor or rechargeable battery for storing energy that it receives.

In some embodiments of the invention, transmitter 62 transmits electromagnetic energy to circuitry 60 and receiver 64 comprises a suitable antenna for receiving the electromagnetic energy. For such embodiments, casing 36 is formed from a material or materials and configured so that it does not prevent receiver 64 from receiving electromagnetic energy. For example, casing 36 may be formed from a non-conducting material or may comprise a portion formed from a conducting material that functions as an antenna. Methods and devices similar to those described in U.S. Pat. No. 5,571,152, the disclosure of which is incorporated herein by reference, may be used for transmitting energy to capsule 24 to power light source 22 and controlling the light source.

Optionally electromagnetic energy is transmitted to capsule 24 at optical frequencies and transmitter 62 comprises a suitable light source for illuminating capsule 24 and receiver 64 comprises a suitable photosensitive receiver, such as a photodiode, for receiving energy transmitted at optical frequencies. In some embodiments of the invention, transmitter 62 comprises an acoustic transmitter for transmitting acoustic energy to capsule 24, and receiver 64 comprises an acoustic transducer for receiving the transmitted acoustic energy. Optionally, at least one acoustic transducer 30 is controllable to transmit acoustic waves and functions as the acoustic transmitter. Methods and devices for powering and controlling a light source implanted in a body using acoustic energy transmitted to the light source from a source external to the body are described in U.S. Pat. No. 6,622,049, the disclosure of which is incorporated herein by reference, and similar methods and devices may be used in the practice of the present invention.

Circuitry 60 controls light source 22 to transmit pulses of light through aperture 38 at, at least one wavelength of light that is absorbed by glucose to illuminate tissue region 53 and stimulate photoacoustic waves in the region. Optionally, at least one of the wavelengths is a signatory wavelength of glucose. Light at glucose signatory wavelengths is generally strongly absorbed by water and attenuates rapidly as a function of propagation distance in living tissue. As a result, light 34 provided by light source 22 at a signatory wavelength attenuates rapidly as a function of distance from aperture 38 and therefore intensity of photoacoustic waves stimulated at a given distance from the aperture by the light falls off rapidly as the given distance increases. However, because of growth of vascularized tissue in region 53 in close proximity to aperture 38, which is stimulated by bio-promoting layer 52, signatory light 34 that exits the aperture interacts with blood and/or interstitial fluid comprising glucose before the light is strongly attenuated. Photoacoustic waves 32, which are stimulated by the interaction therefore tend to have their origins, represented by asterisks 65, in and in a close neighborhood of bio-promoting layer 52 and are responsive to glucose concentration in the patient's blood and interstitial fluid.

At least one acoustic transducer 30 receives energy in photoacoustic waves 32 and generates signals responsive thereto that it transmits to a processor 66, optionally comprised in control unit 28. Processor 66 processes the signals using any of various methods known in the art to determine glucose concentration of blood and interstitial fluid in region 53 and thereby of the patient. Processing the signals generally comprises using any of various methods known in the art and calibration data acquired for glucometer 20. Calibration data comprises data relating to effects of interaction of light 34 with material from which membrane 50 is formed on the generation and characteristics of photoacoustic waves 32. Calibration data is optionally acquired by comparing glucose measurements provided by glucometer 20 to glucose measurements provided by any of various reliable invasive techniques known in the art.

By way of example, in some embodiments of the invention, circuitry 60 controls light source 22 to illuminate tissue 53 with pulses of light 34 at a plurality wavelengths to stimulate photoacoustic waves 32 in the tissue. Optionally, at least one of the wavelengths is a signatory wavelength for glucose in interstitial fluid and/or blood. Signals generated by at least one transducer 30 responsive to photoacoustic waves 32 stimulated by light 34 at each of the wavelengths are processed using a suitable multivariate method known in the art to assay glucose in region 53. Since, optionally, at least one of the wavelengths is a signatory wavelength of light for glucose, the plurality of wavelengths used to assay glucose in region 53 may be a relatively small plurality.

For example, in some embodiments of the invention, circuitry 60 controls light source 22 to illuminate region 53 with light at a first wavelength that is a glucose signatory wavelength for interstitial fluid, for example 9.66 microns. At 9.66 microns absorption of light in region 53 and therefore generation of photoacoustic waves 34 in the region are due substantially only to concentrations of glucose and water in the region. (Generation of photoacoustic waves resulting from interaction of the light with material in membrane 50 is accounted for by the calibration data.) In addition, light source 22 is optionally controlled to illuminate region 53 with light 34 at a second wavelength, for example 1.44 microns, for which the absorption coefficient of the light is substantially equal to the absorption coefficient for water. Stimulation of photoacoustic waves 34 in tissue region 53 by light at the second wavelength is therefore due substantially only to concentration of water in the region. Signals responsive to the photoacoustic waves are optionally used to determine concentration of water in the region. The determination of water concentration in tissue 53 responsive to photoacoustic waves stimulated by light at the second wavelength enables absorption of light 34 at the first wavelength due to glucose in the tissue, and therefore an assay of the patient's glucose, to be determined responsive to photoacoustic waves stimulated by light at the first wavelength.

By way of another example, light at first and second wavelengths 9.66 and 9.02 microns may be used to stimulate photoacoustic waves in region 53. Light at 9.02 microns is a signatory wavelength of glucose that stimulates photoacoustic waves in the region due to interaction of the light with substantially only glucose, water and hemoglobin. (As noted above, generation of photoacoustic waves resulting from interaction of the light with material in membrane 50 is assumed to be accounted for by the calibration data.) In accordance with an embodiment of the invention region 53 is also illuminated with light at a third wavelength, for example 0.810 microns, that is a signatory wavelength of hemoglobin. Light at 0.810 microns generates photoacoustic waves in region 53 substantially only as a result of interaction of the light with hemoglobin in the region. In accordance with an embodiment of the invention photoacoustic waves stimulated by light at the three wavelengths is used to assay the patient's glucose.

It is noted that practice of the present invention is not limited to the use of two or three wavelengths and/or signatory wavelengths. Light source 22 may be controllable by circuitry 60 to provide light at a suitable plurality of different signatory and/or non-signatory wavelengths to stimulate photoacoustic waves from which to determine glucose concentration. Any of various multivariate methods known in the art may be used to process signals generated responsive to the photoacoustic waves to determine glucose concentration.

In some embodiments of the invention, changes in an acoustic property of tissue in proximity to aperture 38 as a result of illumination by light 34, rather than photoacoustic waves generated in the tissue by light 34 are used to assay the patient's glucose. For example, prior to and subsequent to and/or during illumination of the tissue by light 34, acoustic transducer 30 is controlled to transmit ultrasound that is incident on tissue region 53. Some of the incident ultrasound is reflected back to the at least one transducer from aperture 38. A difference in the ultrasound received before and after and/or during illumination is used to assay glucose in region 53. The measurement of changes in acoustic properties of a tissue region generated by illuminating the tissue region and the uses of the measurements to determine glucose concentration in the region is described in U.S. patent application Ser. No. 10/312,300, filed by some of the same inventors of the present invention, the disclosure of which is incorporated herein by reference.

In some embodiments of the invention glucometer 20 is controlled to assay glucose responsive to photoacoustic waves generated by light 34 and responsive to changes in acoustic properties generated by illumination of region 53 with the light. Optionally, a glucose assay “reported” by glucometer 20 is determined responsive to both the photoacoustic assay and the acoustic property change assay. In some embodiments of the invention the two types of assays are used to monitor and update calibration data for glucometer 20.

Control unit 28 is optionally equipped with a suitable input and output device or devices known in the art for displaying, transmitting and/or storing glucose assays and receiving instructions from a user and responding to received instructions. In some embodiments of the invention, assays provided by glucometer 20 are transmitted to a controller that controls a device, such as an insulin pump, for delivering medication to the patient.

Capsule 24 is inserted below skin 26 of the patient using any of various methods and devices known in the art. Optionally, capsule 24 and its contents are fabricated, using methods known in the art so that the capsule is small enough to be conveniently inserted into a patient's body using a catheter or suitable syringe and needle.

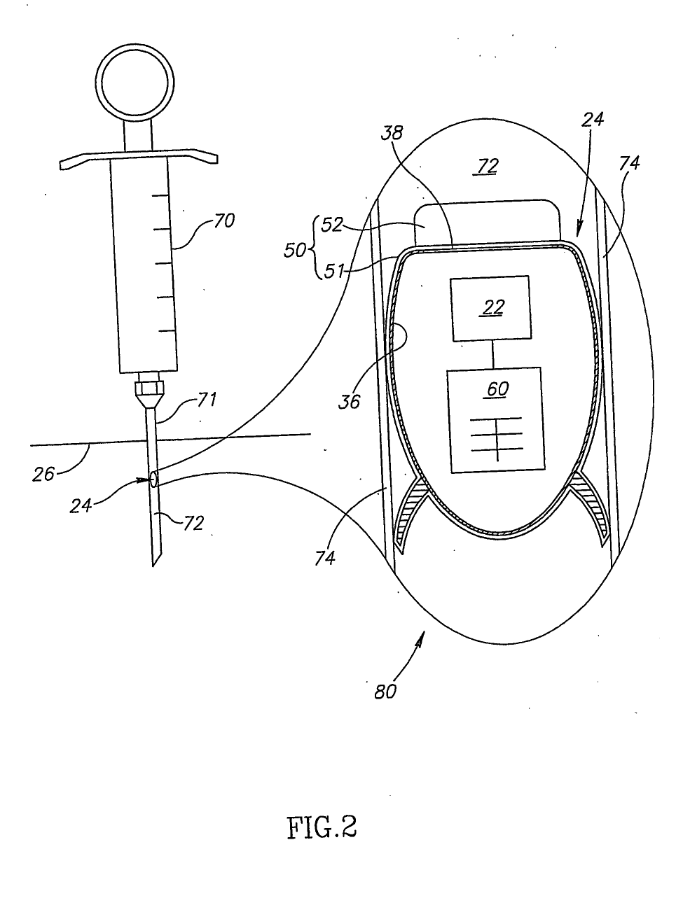

FIG. 2 schematically shows a syringe 70 and needle 71 having a lumen 72 being used to insert capsule 24 below skin 26 of the patient. Capsule 24 is suspended in a suitable carrier liquid, for example a saline solution and drawn up into lumen 72 of needle 71 together with a quantity of saline solution so that the capsule is optionally oriented with aperture 38 of the capsule facing syringe 70. Orientation of capsule 24 in needle 71 is optionally achieved by pre-loading the capsule into a suitable guide tube which is suspended in the carrier liquid and then drawing up the carrier liquid and capsule through the guide tube. FIG. 2 shows capsule 24 after it has been drawn up into lumen 72 of needle 71. As the carrier liquid is expelled from syringe 70, capsule 26 is propelled out of needle 71 and into the patient's body. Inset 80 shows an enlarged view of a portion of lumen 72 and capsule 24. Anchor stubs 40 are optionally formed from an elastic material and are forced to fold towards the body of capsule 24 by wall 74 of needle 71 when the capsule is drawn into lumen 72. When capsule 24 exits the lumen, anchor hooks 40 splay outward into the positions in which they are shown in FIG. 1.

Whereas in FIGS. 1 and 2 anchor stubs 40 are optionally used to stabilize position of capsule 24 after the capsule is introduced below skin 26, other methods of stabilizing the position of capsule 24 may be used. For example, the outside surface of capsule 24 may be formed with a plurality of ridges that increase friction between the capsule and body tissue. Optionally, capsule 24 does not require special stabilization features. Location of the capsule may be sufficiently stable without such special features for proper operation of glucometer 20. For example, the position of capsule 24 after it is introduced below skin 26 may be stable as a result of body processes, such as natural encapsulation provided by a foreign body response, the characteristics of a tissue region into which the capsule is introduced or as a result of capsule size and/or shape.

In the examples shown in FIGS. 1 and 2 photoacoustic waves are generated in membrane 50 and tissue regions in the neighborhood of the membrane. In some embodiments of the invention, membrane 50 is formed from materials that substantially do not interact with light 34 and/or is made so thin that it does not substantially absorb and attenuate light 34. As a result, a relatively small portion of photoacoustic waves 32 is stimulated in bio-promoting layer 52 while a relatively large, or major portion of the photoacoustic waves, is stimulated by the light in region 55. In some embodiments of the invention, interaction of membrane 50 with light 34 is so small that photoacoustic waves 32 stimulated in bio-promoting layer 52 may be ignored in assaying the patient's glucose. For such embodiments, effects of interaction of light 34 with material in bio-sealing layer 51 and vascularized tissue therein is reduced and calibration data and its use in assaying the patient's glucose levels simplified. And the patient's glucose is assayed responsive to photoacoustic waves stimulated in tissue substantially unaffected by the presence of capsule 24.

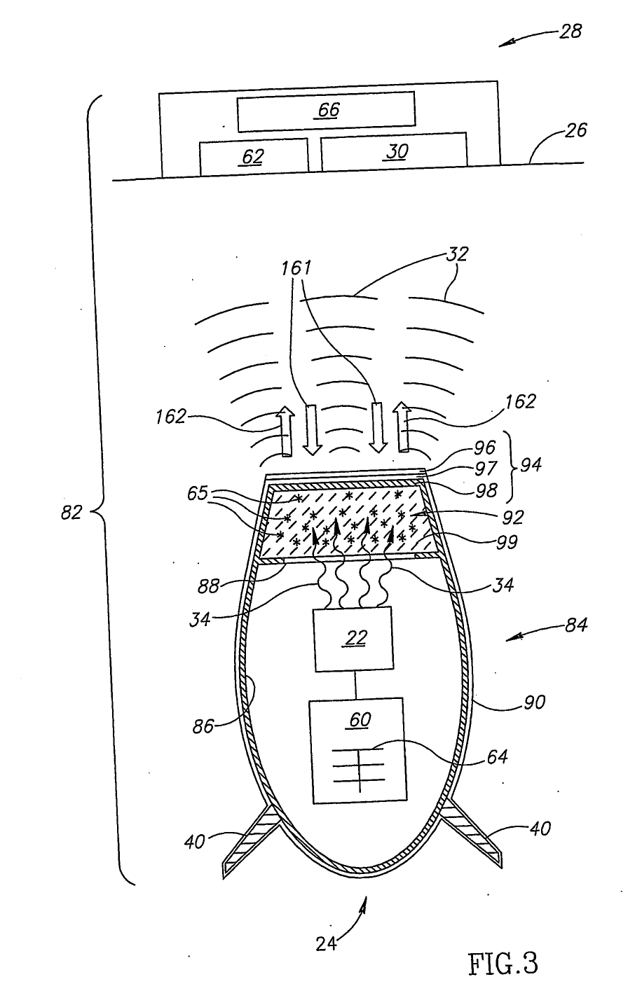

FIG. 3 schematically shows another glucometer 82 assaying a patient's glucose, in accordance with an embodiment of the invention. Glucometer 82 comprises a light source 22 encapsulated in a capsule 84 which is implanted below skin 26 of the patient and a control unit 28.

Capsule 84 is similar to capsule 24 shown in FIGS. 1 and 2 and has a casing 86 comprising light source 22 and associated control circuitry 60. Casing 86 has an optical aperture 88 through which light 34 provided by light source 22 is transmitted. Optical aperture 88 and casing 86 are optionally covered with a bio-sealing layer 90 that protects the contents of the capsule from damage by body processes. Unlike capsule 24, capsule 84 is formed so that it comprises a “test volume” 92 illuminated by light 34.

Test volume 92 is bounded by a biocompatible membrane 94 and optionally by a portion of casing 86. Membrane 94 is designed to moderate or prevent a foreign body response thereto by the patient's body and to be sufficiently permeable to components of interstitial fluid so that interstitial fluid and components thereof, and in particular glucose, diffuse relatively easily through the membrane. Block arrows 161 and 162 schematically indicate diffusion of interstitial fluid and components thereof through membrane 94, into and out of test volume 92 respectively. Test volume 92 is therefore filled with interstitial fluid 99 and a difference between concentration of glucose in interstitial fluid 99 and an equilibrium concentration of glucose in interstitial fluid 99, which might for example occur as glucose concentration in the patient's body changes, has a relaxation time that is generally relatively moderate. Additionally or alternatively, the relaxation time is optionally calibrated. As a result, concentration of glucose inside test volume 92 is useable as representative of the concentration of glucose in interstitial fluid outside of the test volume.

As a result concentrations of glucose and other components of interstitial fluid 99 are useable as representative of the concentrations of glucose in interstitial fluid outside of test volume 92.

Membrane 94 is optionally designed and formed using methods and materials similar to those described in US Patent Application Publication US 2003/0023317 and optionally comprises a bio-promoting layer 96 and a bio-sealing layer 97. Bio-promoting layer 96 promotes in-growth of vascularized tissue in the bio-promoting layer. Bio-sealing layer 97 is substantially impervious to cells and tends to prevent formation of a barrier cell layer thereon but is permeable to interstitial fluid and glucose therein. Optionally membrane 95 comprises a substrate membrane 98 on which layers 96 and 97 are formed. Substrate membrane 98 is also permeable to interstitial fluid and glucose and functions to provide structural strength to membrane 95. Substrate membrane 98 may be formed using methods known in the art from materials comprising for example cellophane, cellulose, cuprophane and/or polyethersylphone.

Glucometer 82 assays glucose in interstitial fluid 99 in test volume 92 to determine the patient's glucose level. To perform an assay of interstitial fluid 99, controller 60 controls light source 22 to illuminate the interstitial fluid with light 34 at a plurality of wavelengths. Optionally, at least one of the wavelengths is a signatory wavelength of glucose in interstitial fluid. Light 34 when it enters test volume 92 stimulates photoacoustic waves 32 having origins at locations 65 in interstitial fluid 99. Signals generated by transducer 30 responsive to acoustic energy in photoacoustic waves 32 are processed by processor 66 to determine glucose concentration of interstitial fluid 99. Suitable calibration data is used in the determination of the glucose concentration in the interstitial fluid and to relate it to the patient's glucose level.

Because test volume 92 comprises substantially only interstitial fluid 99 and components thereof, a number of different analytes that light 34 interacts with in stimulating photoacoustic waves 32 is generally less than a number of different analytes the light would interact with in vascularized tissue. For example, interstitial fluid 99 does not generally contain hemoglobin. As a result, it is generally possible to assay the patient's, glucose with glucometer 82 using a number of different wavelengths of light 34 that is less than a number that might otherwise be required.

For example, in vascularized tissue it is possible to assay glucose in accordance with an embodiment of the invention using light 34 at glucose signatory wavelengths 9.66 microns and 9.02 microns to stimulate photoacoustic waves 32 in the tissue. But in addition, as described above, because light at wavelength 9.02 microns interacts relatively strongly with hemoglobin in vascularized tissue, light 34 at an additional wavelength, such as 0.811 microns, is generally also used to stimulate photoacoustic waves 32 in the tissue. The photoacoustic waves stimulated by light at the additional wavelength provide information useable to estimate the tissue's hemoglobin concentration. However, using glucometer 82, since the glucometer measures glucose concentration in interstitial fluid 99, which does not comprise hemoglobin, it is possible to provide an assay of the patient's glucose using light at wavelengths 9.66 and 9.02 microns only.

In some embodiments of the invention, membrane 94 is formed to filter out analytes in interstitial fluid of the patient's body that at wavelengths of light used to assay glucose in interstitial fluid in test volume 92 might otherwise interfere with the assay. For example, if light at wavelengths that are used to assay glucose also interact with albumen it can be advantageous for membrane 94 to be substantially impermeable to relatively large molecules such as albumin. Interstitial fluid in test volume 92 would therefore be relatively devoid of albumen and glucose assays provided by glucometer 82 simplified and/or improved.

It is noted that whereas in the above description of exemplary embodiments, glucose is assayed, the present invention is not limited to assaying glucose. Methods and devices similar to those used for assaying glucose, in accordance with an embodiment of the invention may, with appropriate choice of wavelengths of light provided by light source 22, be used for assaying other analytes present in body tissue. For example, a patient's albumin or lactate may be assayed, in accordance with embodiments of the invention. Albumin has a signatory wavelength at about 6.25 microns and lactate has a signatory wavelength at about 8.81 microns.

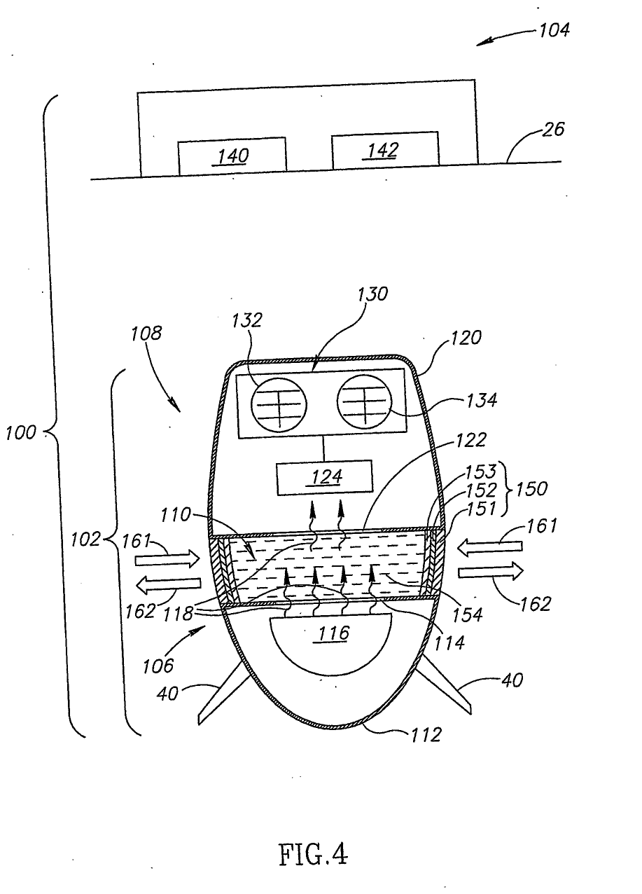

FIG. 4 schematically shows a glucometer 100 determining glucose concentration in a patient's interstitial fluid, in accordance with another embodiment of the present invention. Glucometer 100 comprises a sensor capsule 102 implanted beneath skin 26 of the patient and a controller 104 optionally mounted on the patient's skin. Sensor capsule 102 comprises an optical transmission unit 106 and an optical sensor unit 108 that sandwich between them a test volume 110. Optionally, at least one of optical transmission unit 106 and optical sensor unit 108 are adapted to stabilize sensor capsule 102 in the patient's body. By way of example, in glucometer 100 transmission unit 106 comprises stabilizing anchor stubs 40 similar to those comprised in capsule 24 of glucometer 20 (FIGS. 1 and 2).

Optical transmission unit 106 comprises a casing 112 having an optical exit aperture 114 and a light source 116 controllable to transmit light 118 through the exit aperture and into test volume 110 at at least one wavelength that is absorbed by glucose.

Optical sensor unit 108 comprises a casing 120 having an optical input aperture 122 and a photosensor 124 that senses light transmitted by light source 116 that passes through test volume 110 and enters the optical sensor unit through input aperture 122. Optionally, sensor unit comprises control circuitry 130 for controlling and powering light source 116 and photosensor 124 and receiving signals generated by the photosensor responsive to light 118 that the photosensor receives. Optionally, circuitry 130 is powered by energy that it receives from a transmitter 140, optionally located in controller 104, and comprises a receiver 132 for receiving the energy. Optionally, circuitry 130 comprises a device (not shown) for storing energy, such as a capacitor or rechargeable battery for storing energy that it receives. Optionally circuitry 130 comprises a transmitter 134 for transmitting signals to a receiver 142 optionally located in controller 104.

Test volume 110 is bounded by a membrane 150, similar to membrane 94 (FIG. 3), that is sufficiently permeable to interstitial fluid so that interstitial fluid and components thereof, and in particular glucose, diffuse relatively easily through the membrane. Membrane 150 optionally comprises a bio-promoting layer 151 and a bio-sealing layer 152 optionally formed on a substrate membrane 153. Block arrows 161 and 162 schematically indicate diffusion of interstitial fluid and components thereof through membrane 150, into and out of test volume 110 respectively.

Test volume 110 is therefore filled with interstitial fluid 154 and a difference between concentration of glucose in interstitial fluid 154 and an equilibrium concentration of glucose in the interstitial fluid has a relaxation time that is generally relatively moderate. Additionally or alternatively, the relaxation time is optionally calibrated. As a result, concentration of glucose inside test volume 110 is useable as representative of the concentration of glucose in interstitial fluid outside of the test volume.

To assay the patient's glucose, circuitry 130 controls light source 116 to illuminate test volume 110 with light 118 at a plurality of wavelengths at least one of which is absorbed by glucose. Interstitial fluid 154 in test volume 110 absorbs a portion of incident light 118 and a portion of the light propagates though the test volume, passes though input aperture 122 of optical sensor unit 108 and is incident on photosensor 124. Optionally, thickness of test volume 110 along a direction from light source 116 to photosensor 124 has a value in a range from about 10 microns to about 50 microns. In some embodiments of the invention, the thickness has a value in a range from about 50 to about 150 microns.

Photosensor 124 generates signals responsive to intensity of the incident light and transmits the signals to circuitry 130. Circuitry 130 optionally uses the signals it receives from photosensor 124 to determine attenuation of the light as a result of transmission through test volume 100 and uses the determined attenuation to assay glucose in the interstitial fluid using any of various methods known in the art. Optionally, circuitry 130 transmits the signals it receives from photosensor 124 to controller 104, which optionally processes the signals to assay glucose in interstitial fluid 154.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for assaying an analyte in a body comprising:
at least one light source implanted in the body controllable to illuminate a tissue region in the body with light at at least one wavelength that is absorbed by the analyte and generates thereby photoacoustic waves in the tissue region;
at least one acoustic sensing transducer coupled to the body that receives acoustic energy from the photoacoustic waves and generates signals responsive thereto; and
a processor that receives the signals and processes them to determine a concentration of the analyte in the illuminated tissue region.

2. Apparatus according to claim 1 and comprising.
at least one acoustic transmitting transducer coupled to the body controllable to transmit ultrasound,
a controller adapted to control the at least one transmitting transducer; wherein
the controller controls the transmitting transducer to transmit ultrasound that is incident on the illuminated tissue region and thereafter on the at least one sensing transducer and the processor processes signal generated by the sensing transducer responsive to the ultrasound to determine a change in an acoustic property of the tissue region caused by the illumination and therefrom an assay of the analyte.

3. Apparatus for assaying an analyte in a body comprising:
at least one light source implanted in the body controllable to illuminate a tissue region in the body with light at at least one wavelength that is absorbed by the analyte and generates a change in an acoustic property of the region;
at least one sensing acoustic transducer that generates signals responsive to acoustic energy incident thereon;
at least one transmitting acoustic transducer that transmits ultrasound that is incident on the region and thereafter on the sensing transducer; and
a processor that receives signals generated by the sensing transducer responsive to the incident ultrasound and processes them to determine a measure of the change and therefrom concentration of the analyte.

4. Apparatus for assaying an analyte in a body comprising:
a membrane formed from a material permeable to interstitial fluid in the body and the analyte therein that bounds a volume region in the body, which volume region contains interstitial fluid that permeates through the membrane to enter the volume;
at least one light source implanted in the body that illuminates the volume with light at at least one wavelength that is absorbed by the analyte and generates thereby photoacoustic waves in the volume;
at least one acoustic sensing transducer coupled to the body that receives acoustic energy from the photoacoustic waves and generates signals responsive thereto; and
a processor that receives the signals and processes them to determine a concentration of the analyte in the illuminated volume.

5. Apparatus for assaying an analyte in interstitial fluid in a body comprising:
at least one light source implanted in the body that provides light at at least one wavelength that is absorbed by the analyte;
at least one photosensor implanted in the body that receives light from the at least one light source and generates signals responsive thereto;
a membrane formed from a material permeable to components of interstitial fluid in the body and the analyte that bounds a volume sandwiched between the at least one light source and the at least one photosensor and wherein the light from the at least one light source that reaches the at least one photosensor propagates through the volume; and
circuitry that receives the signals from the at least one photosensor and uses them to provide an assay of the analyte in the body.

6. Apparatus according to claim 5 wherein a gap between the source and the sensor is 10 to 50 micrometers.

7. Apparatus according to claim 5 wherein a gap between the source and the sensor is 50 to 150 micrometers.

8. Apparatus according to any of claims 1-7 wherein the absorption coefficient for light in tissue in the region at a wavelength of the at least one wavelength is substantially equal to the sum of the absorption coefficients of the analyte and at most a relatively small number of additional analytes.

9. Apparatus according to claim 8 wherein the at least one additional analyte comprises a number of analytes less than or equal to three.

10. Apparatus according to claim 8 wherein the at least one additional analyte comprises a number of additional analytes less than or equal to two.

11. Apparatus according to claim 8 wherein the at least one additional analyte comprises one additional analyte.

12. Apparatus according to any of claims 8-11 wherein the at least one additional analyte comprises water.

13. Apparatus according to any of claims 1-12 wherein the at least one implanted light source is encapsulated in a capsule having an aperture substantially transparent to light at the at least one wavelength through which light is transmitted to illuminate the region.

14. Apparatus according to claim 13 and comprising a layer of a biocompatible material overlaying the aperture that promotes vascularization of tissue in close proximity to the aperture and wherein the region comprises the vascularized tissue.

15. Apparatus according to claim 14 wherein vascularization is promoted at distances from the aperture that are less than an extinction length for light at the at least one wavelength.

16. Apparatus according to claim 13 or claim 15 wherein the capsule comprises a receiver for receiving energy to power the at least one light source.

17. Apparatus according to claim 16 wherein the receiver comprises an antenna for receiving electromagnetic energy.

18. Apparatus according to claim 17 and comprising a transmitter external to the body that transmits electromagnetic energy to the antenna.

19. Apparatus according to any of claims 16-18 wherein the receiver comprises an acoustic transducer for receiving acoustic energy.

20. Apparatus according to claim 19 and comprising an acoustic transmitter external to the body that transmits acoustic energy to the acoustic transducer comprised in the receiver.

21. Apparatus according to any of the preceding claims wherein the analyte is glucose.

22. Apparatus according to any of the preceding claims wherein the at least one wavelength comprises a wavelength equal to about 9.66 microns.

23. Apparatus according to any of the preceding claims wherein the at least one wavelength comprises a wavelength equal to about 9.02 microns.

24. Apparatus according to any of the preceding claims wherein the at least one wavelength comprises a plurality of wavelengths.
